(19) United States
(12) Patent Application Publication
(10) Pub. No.: US 2022/0309947 A1
(43) Pub. Date: Sep. 29, 2022

(54) SYSTEM AND METHOD FOR MONITORING AND TEACHING CHILDREN WITH AUTISTIC SPECTRUM DISORDERS

(71) Applicant: NEUROGRESS LIMITED, Sheung Wan (HK)

(72) Inventors: Yulia Olegovna LOBODA, Tomsk (RU); Konstantin Yurievich GORBUNOV, Novosibirsk (RU)

(21) Appl. No.: 17/597,186

(22) PCT Filed: Jun. 22, 2020
(86) PCT No.: PCT/RU2020/050133

§ 371 Date: Dec. 28, 2021

(30) Foreign Application Priority Data
Jul. 10, 2019 (RU) 2019121641

(51) Int. Cl. G09B 19/00; A61B 5/369; A61B 5/00

(52) U.S. Cl. CPC G09B 19/00; A61B 5/369; A61B 5/0006

(57) ABSTRACT

The invention relates to methods and systems for monitoring and teaching children with autistic spectrum disorders and can be used for effectively managing special educational work with children with autistic spectrum disorders (ASDs). According to the invention, the system comprises a remote server, personal computer devices of parents and specialists that are connected by an integrated network to the remote server, and a neuro-interface module for tracking a child's brain activity, said module being placed on the child and being connected by means of the integrated network to the remote server and comprising EEG sensors, wherein the neuro-interface module comprises an accelerometer and a gyroscope, and sensors for detecting a gaze direction, and the remote server is capable of collecting and analyzing visual data about the child's activity.

### FIELD OF THE INVENTION

The invention relates to the field of education, particularly to methods and systems for monitoring and teaching children with autistic spectrum disorders, which methods and systems comprise a teaching robot, a personal computer device of a child, each of which is equipped with a data exchange module for data exchange between the child's computer device and other devices and with external devices by means of an integrated network and can be used for effectively managing special educational work with children with autistic spectrum disorders (ASDs).

The following terms are used in the description:

Server is an electronic device that performs service functions at the request of the client, providing the client with access to certain resources. For the purposes of this description, a server is considered that has a persistent connection to an integrated network configured to transfer data from client devices to the server. The server is configured to process this data and transfer the result of the processing back to the client device.

An integrated network, and also all connections between all modules and blocks include various topologies, layouts and arrangements of network interconnection components, configured to interconnect corporate, global, and local area networks, and include, without limitation, traditional wired, wireless, satellite, optical and equivalent network technologies. Preferably, the Internet is usually used as an integrated network.

User's personal device is any form of computing platform being able to connect to a network, such as an integrated network, and to allow interaction with applications. Illustrative examples of individual client devices include, but are not limited to, stationary and portable personal computers, “smart” cell phones (smartphones), laptop computers including tablet computers, lightweight clients, workstations, and “dumb” terminals connected to the application server, and also various layouts and configurations thereof, that is, both physical devices for interaction in communication systems and virtual devices implemented on programmable computer devices and having a software interface for performing communication functions. Preferably, it is a smartphone (smart phone, that is, a cell phone having the functions of a computer device) with a touchscreen display or a tablet computer or similar devices in the form of “smart” watches, glasses, etc. These devices often are portable and can be carried around.

The following abbreviations are used in the description:

ASD is Autistic Spectrum Disorder.

EEG is electroencephalogram. (From electroencephalography (EEG), which is a section of electrophysiology that studies patterns of the total electrical activity of the brain, registered on the surface of the scalp, and also the method of recording such potentials (in the form of electroencephalograms). Also EEG is a non-invasive method for studying the functional state of the brain by registration of its bioelectric activity).

ABA (Applied behavior analysis). ABA therapy is an intensive training program that builds on behavioral technologies and teaching methods. ABA as a scientific discipline studies the influence of factors of the environment on behavior and manipulates these factors to change human behavior.

Motor activity is the position of the body in space and body movements. As used in the specification, term “motor activity” refers to the detecting child's posture and movements.

State of the Art for the System

Autistic spectrum disorders (ASDs) have a high prevalence in Russia: about 1% of children (letter from the Ministry of Health of the Russian Federation No. 15-3/10/1-2140 from May 8, 2013); with the total number of 31,715,000 children in Russia (according to the Rosstat data at the beginning of 2016), it means that approximately 317,150 (1%) of them live with ASDs. For other countries, this problem is also relevant; according to US (CDC) and UK (NHS) medical statistics, more than 1% of children have ASDs. Social adaptation is an important problem. Children with ASDs generally do not fit into kindergarten and school settings because they lack social behavioral skills and have communication problems. There are also difficulties with learning, because even with the preservation of intellect, it is difficult for such a child to follow the rules dictated by the education system.

There are a number of interrelated problems. First of all, the ASDs syndrome is individual, each child needs a special approach, and therefore, detailed documentation of work with the child and a thorough analysis of this information by each new teacher or psychologist working with this child is needed. Most in demand methodology for working with autistic children in the world is ABA; according to it, a child with ASDs needs 30 hours of lessons each week to achieve a stable positive result. One lesson with a professional teacher costs from 1,500 rubles, and respectively, a month of such lessons costs an amount that is almost 5 times higher than the average salary in the Russian Federation. Another problem is the need for the constant presence of an adult with a child. This entails additional costs for the family; it is necessary either to hire a separate person, or one of the family members should refuse to work.

As a result, about 90% of children with ASDs in Russia do not receive sufficient qualified assistance, they cannot successfully integrate into society, generate value, and benefit from social engagement.

At the moment, lessons for children with ASDs are conducted under the constant supervision of a trained teacher, usually in specialized centers. At the same time, both in the centers and at home, constant supervision of the child is needed so that the child does not harm himself.

Today, one of the most effective methods of correcting autism is behavioral therapy or method of applied behavior analysis, ABA.

At the same time, there are already many platforms for distance learning. These platforms not only simplify the interaction between the student and the teacher, but also provide reporting on the work done and changes in the student's level. Such systems are widespread in various fields, but they do not provide the possibility to monitor the state of the student.

At the moment, there are many tools for monitoring the state of the human in the form of devices for obtaining EEG of the brain (neuro-interfaces), myosensors, motion sensors, and programs for detecting emotions, movements, and visual activity.

However, there is still no way to effectively combine the listed means and techniques so that the child is not overloaded with technical devices, being interested in the process, while the sensors are effectively triggered when a dangerous situation arises.

According to the first aspect, the present invention relates to a system for monitoring and teaching children with autistic spectrum disorders, the system comprises a teaching robot that comprises at least a microprocessor of the robot, which microprocessor is connected to a data exchange module of the robot for data exchange with external devices by means of an integrated network, a personal computer device of a child comprising at least a microprocessor of the computer device of the child, connected to a video camera of the computer device of the child, a display of the computer device of the child, a data exchange module of the computer device of the child for data exchange with external devices by means of the integrated network.

A similar system is described in the RF patent for utility model No. 152572 published on Jun. 10, 2014.

This system is the closest in technical essence and the achieved technical result and is chosen as the prototype of the proposed invention as a device.

The disadvantage of said prototype is the impossibility to solve the problem of carrying out educational work with a child without the physical presence of an adult. The teacher or parent cannot quit the room, leaving the child to study. In the event of a dangerous or undesirable situation, the adult will not receive a notification.

In addition, such a system has the other disadvantages: impossibility for the teacher:
to select a list of tasks for the student, depending on the individual plan,
to track the student's progress and adjust the training plan,
to keep track of learning statistics;
impossibility for the parent:
to select a list of tasks for the student, depending on the individual plan,
to organize independent activities of the child,
to track the progress of the child and adjust the educational plan;
impossibility for developers:
to add new tasks for students on a regular basis,
to create new courses, to assign rights.

Other Problems According to the Current State of the Art:

1. Constant monitoring of the child's state is needed. A child with ASDs is prone to various repetitive states when the child focuses on one object or action. These can be both dangerous states, for example, when the child begins to bang his head on a hard surface, injure himself, etc., or just inhibition of activity, when the child, instead of performing tasks, performs actions of his own choice with surrounding objects, as a result of which efficiency of the lessons drops.

2. An attempt to put a multitude of motion or physiological state sensors on any child, especially a child with ASDs, may entail additional problems. The child can remove the sensors, harm himself with them, and also various negative emotional reactions are possible.

3. Careful selection of ABA tasks and blocks is needed and can be done interactively. When implementing these blocks, the choice of understandable and recognizable images is especially important.

4. For working with children, it is important to make exercises gamified, which means that all tasks in electronic form should be interactive.

5. For the development of behavioral and communication skills, the child needs an interlocutor who quickly reacts to the child's actions and shows an example of physical activity.

### Disclosure of the Invention for the System

The present invention is mainly directed to providing a monitoring and education system for children with autistic spectrum disorders, which system at least reduces at least one of the above disadvantages, namely: to provide the possibility of carrying out educational work with a child without the physical presence of an adult, that is, the possibility of automatic operation of the system with monitoring of the child's state and remote monitoring of states and managing the system, which is the problem to be solved in the invention.

To achieve this goal, the system comprises a remote server comprising at least a microprocessor of the remote server, which microprocessor is connected to a data exchange module of the remote server for data exchange with external devices by means of an integrated network, personal computer devices of parents and specialists connected to the remote server by means of the integrated network, a neuro-interface module for tracking a child's brain activity being placed on the child and being connected to the remote server by means of the integrated network and comprising EEG sensors, wherein the neuro-interface module comprises an accelerometer and a gyroscope, and sensors for detecting a gaze direction located on the child's personal computer device, and the remote server is configured to collect and analyze visual data about the child's activity: facial expressions, gaze direction, motor activity, and is also configured to automatically transmit data about the current and undesirable states of the child to the personal computer devices of parents and specialists.

Due to these advantageous features, it became possible to carry out educational work with a child without the physical presence of an adult, that is, the possibility of automatic operation of the system with monitoring of the child's state and remote monitoring of states and managing the system.

There is a constant monitoring of the child's state, brain activity, emotional state, and actions.

Therefore, it is a triple monitoring system: recognition of the emotion, detection of the body position, data from the gyroscopes, accelerometers, and EEG sensors (general psychophysiological state).

Video cameras monitor the child's state and make it possible to assess whether the child is repeating certain tasks correctly.

In one embodiment of the invention, the remote server is configured to send signals in the form of push notifications with information about the current state of the child accompanied by a sound signal to the personal computer devices of parents and specialists.

Due to these advantageous features, it became possible to send signals in the form of push notifications with information about the current state of the child accompanied by a sound signal to the personal computer devices of parents and specialists.

In another embodiment of the invention, the EEG sensors include five frontal EEG sensors and two behind-the-ear contacts.

Due to this advantageous feature, it became possible to accurately track a child's brain activity.

In another embodiment of the invention, the remote server is configured to process the incoming data in a neural network mode for accurate recognition of the child's emotions.

Due to this advantageous feature, it became possible to more accurately recognize the child's emotions. The neural network receives an image from a web camera installed at the child's workplace, recognizes a face on it, and then recognizes the emotion expressed. In the case of a negative emotional state, the system notifies parents about it.

The short-term goal of the neural network is to track down undesirable states and notify parents and specialists about them.

The long-term goal of the neural network is the formation of datasets for successful diagnosis and further research of ASD syndrome. This refers to EEG datasets configured to calculate and build an individual educational trajectory of the child.

In addition, in another embodiment of the invention, the teaching robot has a drive for movement and/or simulation of movement.

Due to this advantageous feature, it became possible that the robot not only can maintain a conversation with the child (ask questions, give advice, give “feedback” on tasks, answer questions, etc.), but also show various movements and postures.

Therefore, the problem set in this invention is the possibility of automatic operation of the system with monitoring of the state of the child and remote monitoring of states and managing the system. The problem is solved using the above-mentioned features.

State of the Art for the Method

Another aspect of the present invention is a method for monitoring and teaching children with autistic spectrum disorders.

A similar method is described in the description of the operation of the system disclosed in the patent of the Russian Federation for utility model No. 152572 published on Jun. 10, 2014.

This method is the closest in technical essence and the achieved technical result and is chosen as the prototype of the proposed invention.

The disadvantage of said prototype is the impossibility to solve the problem of carrying out educational work with a child without the physical presence of an adult. The teacher or parent cannot quit the room, leaving the child to study. In the event of a dangerous or undesirable situation, the adult will not receive a notification.

In addition, this method has other disadvantages listed above for the system.

Disclosure of the Invention for the Method

Based on this original observation, the present invention is mainly directed to providing a method for monitoring and teaching children with autistic spectrum disorders, in which method a teaching robot is used to communicate with a child, a child's personal computer device is used to give tasks to the child, the device is connected by means of an integrated network to enable data exchange, which makes it possible at least to reduce at least one of the above disadvantages, namely: to ensure the possibility to carry out educational work with a child without the physical presence of an adult, that is, the possibility of automatic operation of the system with monitoring of the child's state and remote monitoring of states and managing the system, which is the problem to be solved.

To achieve this goal, the method comprises the following steps:
to use a remote server, which is connected to other devices by means of an integrated network, wherein the learning tasks for the child are stored on the server,
to use personal computer devices of parents and specialists, which are connected to other devices by means of the integrated network,
to track a child's brain activity using a neuro-interface module placed on the child,
to monitor the body position of the child using an accelerometer and a gyroscope installed in the neuro-interface module,
to monitor the child's emotions using sensors for detecting a gaze direction,
to process all data on the remote server and transmit data about the current and undesirable states of the child from the server to the personal computer devices of parents and specialists.

Due to these advantageous features, it became possible to carry out educational work with a child without the physical presence of an adult, that is, the possibility of automatic operation of the system with monitoring of the child's state and remote monitoring of states and managing the system.

Constant monitoring of the child's state, brain activity, emotional state, and actions is done.

Therefore, it is a triple monitoring system: recognition of the emotion, detection of the body position, data from the gyroscopes, accelerometers, and EEG sensors.

Video cameras monitor the child's state and make it possible to assess whether the child is repeating certain tasks correctly.

In addition, it makes it possible to automatically transmit data about the current and undesirable states of the child to the personal computer devices of parents and specialists.

In one embodiment of the invention, the method comprises sending the push notifications with information about the current state of the child accompanied by a sound signal to the personal computer devices of parents and specialists.

Due to these advantageous features, it became possible to send signals in the form of push notifications with information about the current state of the child accompanied by a sound signal to the personal computer devices of parents and specialists.

In another embodiment of the invention, the incoming data is processed on the remote server using a neural network for accurate recognition of the child's emotions.

Due to this advantageous feature, it became possible to recognize the child's emotions more accurately. The neural network receives an image from a web camera installed at the child's workplace, recognizes a face on it, and then recognizes the emotion expressed. In the case of a negative emotional state, the system notifies parents about it.

The short-term goal of the neural network is to track down undesirable states and notify parents and specialists about them.

The long-term goal of the neural network is the formation of datasets for successful diagnosis and further research of ASDs.

In another embodiment of the invention, the method comprises simulation of movements using a drive of the teaching robot for demonstration of the movements to the child.

Due to this advantageous feature, it became possible that the robot not only can maintain a conversation with the child (ask questions, give advice, give “feedback” on tasks, answer questions, etc.), but also can show different movements and postures.

### BRIEF DESCRIPTION OF THE DRAWINGS

Other distinctive features and advantages of the invention will be obvious from the description given below by way of illustration and not limiting, with reference to the accompanying drawings, in which:

FIG. 1 schematically illustrates layout of the system for monitoring and teaching children with autistic spectrum disorders according to the invention,

FIG. 2 shows the appearance of the neuro-interface,

FIG. 3 schematically illustrates the relationship of the components of the system according to the invention,

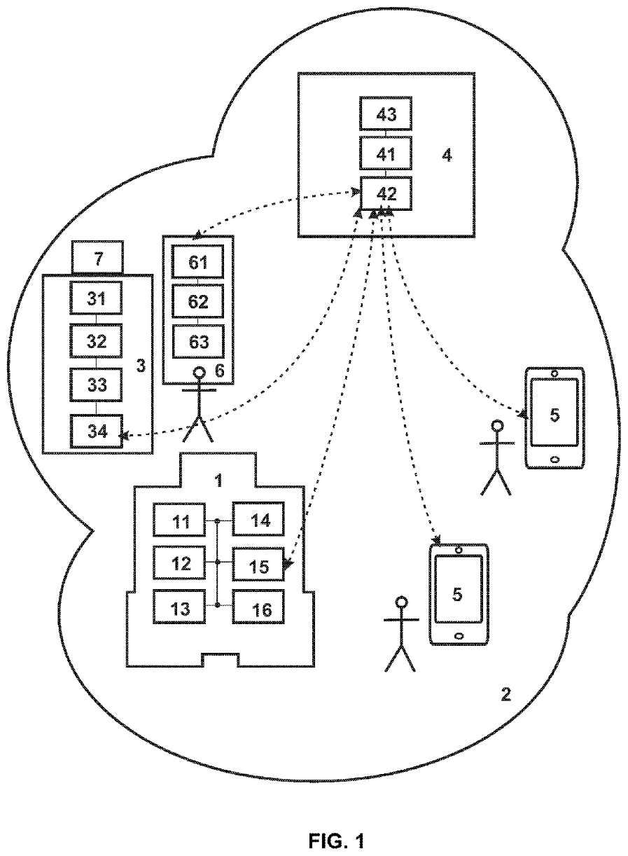

According to FIG. 1-3, the monitoring system, the system for monitoring and teaching children with autistic spectrum disorders comprises a training robot 1 comprising at least a microprocessor 11 of the robot, which microprocessor is connected to an optional video camera 12 of the robot, an optional speaker 13, an optional display 14 of the robot, and a data exchange module 15 of the robot for data exchange with external devices by means of the integrated network 2. The system comprises a personal computer device 3 of a child comprising at least a microprocessor 31 of the computer device of the child, which microprocessor is connected to a video camera 32 of the computer device of the child, a display 33 of the computer device of the child, and a data exchange module 34 of the computer device of the child for data exchange with external devices by means of the integrated network 2. The system comprises a remote server 4 comprising at least a microprocessor 41 of the remote server, which microprocessor is connected to a data exchange module 42 of the remote server for data exchange with external devices by means of the integrated network 2. Also, the remote server 4 has a module 43 for storing databases with all tasks and statistics thereon.

The system also comprises personal computer devices 5 of parents and specialists, connected by means of the integrated network 2 to the remote server 4. Additionally, the system comprises a neuro-interface module 6 for tracking a child's brain activity placed on the child and connected by means of the integrated network 2 with the remote server 4 and comprising EEG sensors 61, wherein the neuro-interface module comprises an accelerometer 62 and a gyroscope 63. The system also comprises sensors for detecting a gaze direction 7 located on the child's personal computer device 3. The remote server 4 is configured to collect and analyze visual data about the child's activity: facial expressions, gaze direction, and motor activity, and is also configured to automatically transmit data about the current and undesirable states of the child to the personal computer devices 5 of parents and specialists.

The remote server 4 is configured to send signals in the form of push notifications with information about the current state of the child accompanied by a sound signal to the personal computer devices 5 of parents and specialists.

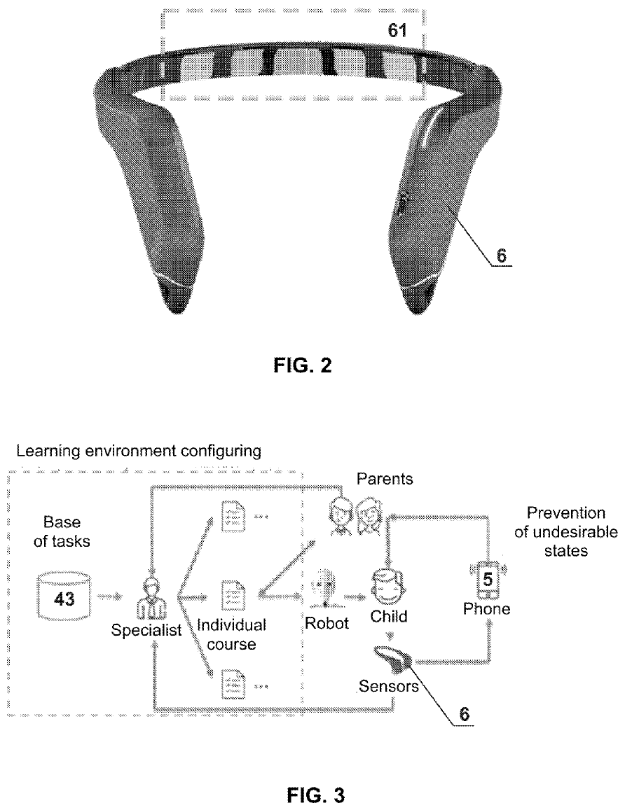

EEG sensors include five frontal EEG sensors and two behind-the-ear contacts. See FIG. 2.

The remote server 4 is configured to process the incoming data in a neural network mode for accurate recognition of the child's emotions.

The teaching robot 1 has a drive 16 for movement and/or simulation of movement. In the general case, the robot may not have a drive, a video camera 12, a loudspeaker 13, and a display 14.

Dotted arrows indicate a connection by means of the integrated network for data exchange.

Implementation of the Invention

The system for monitoring and teaching children with autistic spectrum disorders operates as follows.

Software is installed on the child's personal computer device, which software is a learning environment and a program for processing data about the child's state. A state monitoring application is installed on the personal computer devices 5 of parents and specialists (mobile phone or other suitable device).

In accordance with the recommendations of specialists, blocks of tasks for the child's independent work are selected. The blocks are enabled by means of the learning environment, from the base of exercises 43 connected thereto.

The behavior of the teaching robot 1 is programmed in the tasks. The teaching robot 1 is able to maintain a conversation with the child (ask questions, give advice, give “feedback” on tasks, answer questions, etc.), and also can show various movements and postures.

To monitor the state, the appropriate sensors 6 are put on the child. The psychophysiological state is monitored using a neuro-interface 6. This device tracks brain activity. Normal and undesirable states are programmed. The system can be customized for a particular child, which consists in obtaining the child's EEG data in a normal state and recording them into the database. If there is a significant deviation from the normal state, alerting system is triggered and signals are transmitted to the personal computer devices 5 of parents and specialists.

Motor activity can be monitored using motion sensors 62 and/or 63 or a motion detection program using an image from the camera. The use of sensors is more reliable, but this method is not convenient in the conditions of daily work with a child. Monitoring of the motor activity is needed to complete some gamified tasks, as well as to track undesirable states (excessive motor activity is a warning sign).

An additional means of state monitoring is an emotion recognition program. For its correct operation it is necessary to use a web camera. When strong negative emotions are registered, alerting system is triggered and signals are transmitted to the personal computer devices 5 of parents and specialists.

Monitoring of the child's behavior using a gaze direction tracking system is also provided. It makes it possible to monitor such states when the child stops looking at the robot or display and/or stares into space.

All the means or some of them can be used at once.

Information about the child's state, including notifications about the onset of undesirable or dangerous states, is transmitted to the personal computer devices 5 of parents and specialists.

Reporting on the work done, tasks completed and child's success is implemented by the built-in means of the learning environment.

Management of the learning environment, including access to reporting, can be carried out remotely.

There are four levels of access to the educational platform hosted on the remote server 4:

System administrator. Can add both courses and tasks therein, create users, assign rights. This is the role of the system administrator, an employee of the development company.

Author of the course. Manages the course, can hide unnecessary blocks and customize repetition of the necessary ones, customize the calendar of lessons, and monitor the results. This is a role for an ABA training specialist, an employee of an educational center.

Assistant. Cannot edit the content of the course, but can monitor student's activities, check tasks, give comments. This role is for the parents of the child.

Student. Has access to tasks to be performed. The role is for the child.

The Main Functions of the Platform for the Teacher:
To select a list of tasks for the student, depending on the individual plan.
To track student's progress and adjust the learning plan.
To track learning statistics.

The Main Functions of the Platform for the Parents:
To select a list of tasks for the student, depending on the individual plan.
To organize independent activities of the child.
To track child's progress and adjust the learning plan.

Main Functions of the Platform for the Developers:
Possibility to add new tasks for students on a regular basis.
Creation of new courses, assignment of rights.

The Main Functions of the Platform for the Student:
Constant access to the training platform.
Developing lessons according to the individual plan under the supervision of the teacher.
Constant online monitoring by parents and/or specialists.

Specific Example of Operation

Tasks are divided into seven modules in accordance with the technologies adopted in the ABA therapy, each of the blocks has its own learning goal and is implemented as a separate software set of tasks:

1. Copying Movements

Movements, which the student should repeat, are shown to the student. Movements include actions with objects and selection from several objects, successive touching of objects, and also small motor, facial, and articulatory movements.

There are three levels of difficulty of actions with objects.
Positional simulation. For example, putting a cube in a cup is easier than tapping a cube on a table.
Actions with objects. For example, knock on the table, wave in the air.
Movements with a change of an object. (For example, the first movement with a cube, the second movement with a ball)
Demonstration of more complex actions (for example, assembling a building kit) without specific instructions; the child must repeat the actions himself

Graphomotor imitation (drawing, writing, tracing patterns, etc.).

A hand that draws any lines, shapes, letters, etc. is shown to the child, and the child is invited to repeat these actions.

Demonstration of movements is done by the robot 1 (commands are sent to the robot 1 from the server of the educational platform 4) or via a video clip on the platform (web application).

2. Vocal and Verbal Imitation

Sounds, words, and phrases of different lengths are presented to the student. The student is invited to repeat them. It is implemented in the form of web application.

3. Receptive Speech

Training of a passive vocabulary. Images of objects are presented to the student and each of them is called aloud (a noun vocabulary is formed). Then actions are presented and voiced (a vocabulary of verbs is formed). Then adjectives (colors, shape, size, etc.) and their antagonistic pairs (big-small, dry-wet, etc.) are presented and voiced. Then combinations (noun+verb, noun+adjective, noun+verb+adjective). It is implemented in the form of web application with the artificial intelligence (AI) complex connected for processing audio data. Microphone and loudspeakers are required.

4. Naming

An object is shown to the child, and the child should to name it. The system recognizes and analyzes what the student said. At the next level, the child must name the movements. It is implemented in the form of web application with artificial intelligence (AI) complex connected for processing audio data. A microphone is required.

5. Expressive Speech.

A picture is shown to the child, and the child should to describe it and answer questions about it. It is important that the child should not repeat the phrases that accompanied these pictures at the previous levels. For the successful completion of the task, the child must answer the question correctly. For example, the picture shows a white dancing hare. At the previous step, the child called this picture “White hare”, “Hare is dancing”, “White hare is dancing”. Now the child is asked questions: “Who is this?”, “What is he doing?”, “What color is he?”.

The sequence of pictures and questions is not obvious to the child.

6. Development of the Visual and Cognitive Sphere

Sorting images into categories. Mosaic, puzzles. Tasks aimed at developing the skill of quick recognition and memorization of images. Continuation of logical sequences. Story sequences, restoring the sequence of events. It is implemented in the form of web application.

7. Development of Playing Skills

The use of substitute items. Mastering the concept of transferring the move, playing by the simplest rules. Collecting puzzles together. Performing actions on a condition (for example, the child should click on a ball when his name is called). Board games: dominoes, dice. Sea battle, chess at a higher level. It is implemented in the form of web application.

Each module has several levels of difficulty. In each lesson, tasks from several blocks can be used, but it is necessary to maintain a balance of complexity and take into account the child's capabilities.

### INDUSTRIAL APPLICABILITY

The proposed system for monitoring, system for monitoring and teaching children with autistic spectrum disorders can be implemented in practice by a specialist and, when implemented, provides the achievement of the claimed result, which allows to conclude that the criterion of “industrial applicability” for the invention is met.

In accordance with the proposed invention, a pilot system for monitoring, system for monitoring and teaching children with autistic spectrum disorders has been produced.

ROBOTIS MINI was used as a training robot for testing.

Technical parameters of the ROBOTIS MINI robot:
Controller: OpenCM9.04-C
Control interface: Bluetooth-module BT-210
Programming interface: COM port
Power supply: 2 Li-Ion batteries LB-041
Drive mechanisms: 16 servos DYNAMIXEL XL-320
Drive connection interface: DYNAMIXEL TTL Bus (UART)
Dimensions: 27 cm×35.5 cm×9.5 cm

The neuro-interface Muse was also used for testing, which is a single-channel, non-invasive EEG interface equipped with seven sensors, including five frontal sensors and two behind-the-ear contacts. This provides an excellent signal with minimal noise.

The data captured by the electrodes is the electrical activity of neurons. The formation of patterns of EEG signals is manifested at the moments when a significant number of neurons are synchronized and form a significantly high electrical activity in one period, which can be registered on the surface of the human head.

Therefore, the system receives information about the potential difference between the original raw EEG signal (main electrode) and zero point (reference electrode).

Technical characteristics of the microcontroller used:

The neuro-interface is based on the microcontroller PIC24 (peripheral interface controller) developed by the American company Microchip Technology Inc. The received signal from the sensors is processed and the positions of concentration and relaxation are established, with all EEG data being interpreted and sent to the output.

A triple monitoring system was used to monitor the child's state for testing purposes. The system used two cameras: a web camera on the child's personal computer device and another camera in the room, which gives the maximum view (a camera of the robot can be used), and also a neuro-interface comprising an EEG sensor, a gyroscope, and an accelerometer.

Emotional State Control

A simple high-precision neural network was used to determine the emotional state of the child based on video from the camera. To create the neural network, we used openCV and Keras, and the fer2013 dataset was taken to train it.

The neural network received an image from the web camera installed at the child's workplace, recognized a face on it, and then recognized the emotion expressed. In the case of a negative emotional state, the system notified the parents about it.

Detection of the Body Position

Detection of the body position was carried out based on the data from cameras using recurrent neural network.

Patterns of brain activity were detected based on the data received from the neuro-interface. This includes data on brain activity, body position and movements of the head.

As a result of the test operation of the system for monitoring and teaching children with autistic spectrum disorders, it was found that it makes it possible at once:
To provide the child with a wide range of ABA tasks for development of speaking and behavioral skills according to the ABA programs.
To control remotely the psychoemotional state of the child during lessons.
The possibility for the sensors to effectively trigger when a hazardous situation arises.
To vary the content according to the current state of the child.
To analyze the results of the child's work with the complex.
To implement reporting on the work done.
The claimed system and method make it possible to optimize educational work with children with ASDs for the children themselves and those responsible for them (parents, teachers).
The claimed system and method are designed for children of different ages with various manifestations of ASDs.

Therefore, the claimed system and method solve the problem set out and provide the achievement of the technical result, namely: the possibility of working with a child without the physical presence of an adult, that is, the possibility of automatic operation of the system with monitoring of the child's state and remote monitoring of states and managing the system.

An additional useful technical result of the claimed invention is that the invention provides:
the possibility that the child is not overloaded with technical devices,
the child being interested in the process.

Furthermore:
Independent work of the child is possible.
The program can be customized for each child.
The work of teachers and psychologists becomes easier and more efficient.
The claimed system and method make it possible to carry out lessons at home. At the same time, a specialist of an educational institution still has the opportunity to analyze the results of the child's work on the basis of built-in reporting systems (if needed).
Not only external changes in the child's behavior can be observed, but also psychophysiological state of the child can be monitored.

The claimed system and method can also be used as an educational platform for training the communication and social skills of any child, not only having autistic spectrum disorders; they can also be used not only for organizing work, but even for resting any child.

We claim:

1. A system for monitoring and teaching children with autistic spectrum disorders, comprising:
a teaching robot comprising at least a microprocessor of the robot, which microprocessor is connected to a data exchange module of the robot for data exchange with another device and with external devices by means of an integrated network,
a personal computer device of a child comprising at least a microprocessor of the computer device of the child connected to a video camera of the computer device of the child, a display of the computer device of the child, a data exchange module of the computer device of the child for data exchange with another device and with external devices by means of the integrated network,
wherein the system comprises
a remote server comprising at least a microprocessor of the remote server, connected to the data exchange module of the remote server for data exchange with external devices by means of the integrated network
personal computer devices of parents and specialists connected to the remote server by means of the integrated network,
a neuro-interface module for tracking a child's brain activity placed on the child and connected by means of the integrated network to the remote server and comprising EEG sensors, wherein the neuro-interface module comprises an accelerometer and a gyroscope,
sensors for detecting a gaze direction located on the personal computer device of the child, wherein
the remote server is configured to collect and analyze visual data about the child's activity: facial expressions, gaze direction, and physical activity, and is also configured to automatically transmit data about the current and undesirable states of the child to the personal computer devices of parents and specialists.

2. The system of claim 1, wherein the remote server is configured to send signals in the form of push notifications with information about the current state of the child accompanied by a sound signal to the personal computer devices of parents and specialists.

3. The system of claim 1, wherein the EEG sensors include five frontal EEG sensors and two behind-the-ear contacts.

4. The system of claim 1, wherein the remote server is configured to process the incoming data in a neural network mode for accurate recognition of the child's emotions.

5. The system of claim 1, wherein the teaching robot has a drive for movement and/or simulation of movement.

6. A method for monitoring and teaching children with autistic spectrum disorders comprising the following:
a teaching robot is used to communicate with a child,
a personal computer device of a child is used to give tasks to the child,
the device is connected by means of an integrated network for data exchange,
wherein the method comprises the following steps:
a remote server is used, which is connected by means of the integrated network to other devices, with all the learning tasks for the child stored on the server,
personal computer devices of parents and specialists are used, which are connected to other devices by means of the integrated network,
a child's brain activity is tracked using a neuro-interface module placed on the child,
the body position of the child is monitored using an accelerometer and a gyroscope installed in the neuro-interface module,
the child's emotions is monitored using sensors for detecting a gaze direction,
all data are processed on the remote server with transmitting data about the current and undesirable states of the child from the server to the personal computer devices of parents and specialists.

7. The method of claim 9, wherein the method comprises sending push notifications with information about the current state of the child accompanied by a sound signal to the personal computer devices of parents and specialists.

8. The method of claim 9, wherein the method comprises processing the incoming data on the remote server in a neural network mode for accurate recognition of the child's emotions.

9. The method of claim 9, wherein the method comprises simulation of movements for demonstration to the child using a drive of the teaching robot.
